(19) United States
(12) Patent Application Publication
(10) Pub. No.: US 2025/0213605 A1
(43) Pub. Date: Jul. 3, 2025

(54) PROPHYLAXIS AND THERAPY FOR VASCULAR COMPLICATIONS OF DIABETES WITH IMMUNE BOOSTING

(71) Applicant: Kumarpal A. SHAH, Searingtown, NY (US)

(72) Inventor: Kumarpal A. SHAH, Searingtown, NY (US)

(21) Appl. No.: 18/401,379

(22) Filed: Dec. 30, 2023

(51) Int. Cl. A61K 31/795; A61P 9/00

(52) U.S. Cl. CPC A61K 31/795; A61P 9/00

(57) ABSTRACT

Prophylaxis, immune boosting and therapy for vascular complications of diabetes employing various formulations of sulfonic polymers to target and control the activation loop of complement system at C3 level. The molecular targets identified and successfully inhibited are Factor B, Factor D, Factor H. First, this contributes to the inhibition of cross talk pathways in classical and lectin system pathways as well as the C3a and C5a interactions with their receptors. The cross talk pathways inhibit inflammation, oxidative stress, fatty acid synthesis and fibrosis. Secondly, it further inhibits downstream pathways of C5b-9 and also coagulation and thrombotic cascade. Both pathways cause micro vascular and macro vascular complications of diabetes. Thirdly, patient safety is enhanced by Double inhibition of Factor H and Factor D. This reduces adverse effects mediated by individual inhibition of Factor H and Factor D and in addition it has immune boosting effect by targeting immune evasion.

### BACKGROUND OF THE INVENTION

### 1. Field of the Invention

The invention relates to prophylaxis and therapy for vascular complications of diabetes with immune boosting.

### 2. The Prior Art

The recent evidences indicate that the conventional methods used for the prevention and control of complications of diabetes do not target complement system. It is this web of inflammation that contributes to major vascular complications of diabetes.

To understand the importance of targeting complement system-One should carefully study Complement system and its working as explained in this educational video link www.youtube.com/watch?v=BSypUV6QUNw.

In above video, the critical point we wish to emphasize is how dysfunctional complement system cause inflammation where by analogy the stimulation of one match stick ignites fire by igniting entire match box. The match box analogy is used to ignite multi model stimulation of adverse complement paths that contribute to vascular complications in diabetes and weaken immune system function in diabetes patient.

Tan S. M. et al. in “Forum Review Article: The Complement Pathway: New Insights into Immuno metabolic Signaling in Diabetic Kidney Disease” published their findings in ANTIOXIDANTS & REDOX SIGNALING Volume 37, Numbers 10-12, 2022, the entire contents of which are incorporated herein by reference thereto. They thoroughly reviewed the preclinical evidences and highlighted the importance of preclinical evidences and proposed the need for future directions to target complement pathway in diabetic kidney diseases (DKD) to reduce the burden of microvascular diseases in diabetes.

The importance of above studies are highlighted in the statistics of Diabetes and its social economic burden posed by vascular complications of diabetes.
a. Statistics of Diabetes: (CDC Study: According to Center for Disease Control (CDC) About 38 million people have diabetes. That's about 1 in every 10 people. 1 in 5 don't know they have diabetes.
b. CDC has made aggressive efforts in recent years to identify risk factors for future diabetes. According the Centers for Disease Control (CDC) currently there are 98 M million Americans or one in 3 adults have pre diabetes that is identified as future risk for diabetes and its complications) (www.cdc.gov/diabetes/library/socialmedia/infographics.html).
c. Economics of Diabetes: The diabetes has $413 billion total medical costs and lost work and wages for people with diagnosed diabetes. Risk of early death for adults with diabetes is 60% higher than for adults without diabetes. Medical costs for people with diabetes are more than twice as high as for people without diabetes. People who have diabetes are at higher risk of serious health complications viz blindness; kidney failure; heart disease; stroke; loss of toes, feet, or legs

Parker E. D, et al. did a comprehensive study of “Economic Costs of Diabetes in the U. S. in 2022” in Diabetes Care, ADA Statement (doi.org/10.2337/dci23-0085), the entire contents of which are incorporated herein by reference thereto. Diabetes poses a substantial burden on society in the form of higher direct medical costs, lost productivity, premature mortality, and intangible costs in the form of reduced social connectivity and quality of life. The total estimated cost of diagnosed diabetes in the U.S. in 2022 is $412.9 billion, including $306.6 billion in direct medical costs and $106.3 billion in indirect costs attributable to diabetes. For cost categories analyzed, care for people diagnosed with diabetes accounts for 1 in 4 health care dollars in the U. S. 61% of which are attributable to diabetes. On average people with diabetes incur annual medical expenditures of $19,736, of which approximately $12,022 is attributable to diabetes. People diagnosed with diabetes, on average, have medical expenditures 2.6 times higher than what would be expected without diabetes. Glucose-lowering medications and diabetes supplies account for 17% of the total direct medical costs attributable to diabetes. Major contributors to indirect costs are reduced employment due to disability ($28.3 billion), presenteeism ($35.8 billion), and lost productivity due to 338,526 premature deaths ($32.4 billion).

Saeedi P et al. studied “Global and regional diabetes prevalence estimates for 2019 and projections for 2030 and 20245: Results from the International Diabetes Federation Diabetes Atlas 9th edition, Diabetes Research and Clinical Practice 157 (2019) 107843, doi.org/10.1016/j.diabres.2019.107843 0168-8227, 2019 Published by Elsevier B.V., the entire contents of which are incorporated herein by reference thereto. Accordingly, the global prevalence of diabetes is estimated to be 9.3% (463 million people) rising to 10.2% (578 million) by 2030 and 10.9% (700 million) by 2045.

The vascular complications of diabetes substantially contribute to the high percentage of above cost of diabetes for which there is no simple solutions.

Several drug companies have drugs that are applicable to prevent diabetes and its complications.

The missing component is targeting “Inflammatory Immune Pathogenesis” of diabetes complications. Considerable work has been done in developing this hypothesis. Forbes J. M. and Cooper M. E. reviewed “MECHANISMS OF DIABETIC COMPLICATIONS” and published their findings in Physiol. Rev. 93:137-1888, 2013 (doi: 10,1152/physrev.00045.2011), the entire contents of which are incorporated herein by reference thereto. After reviewing various mechanisms of complications of diabetes they concluded that as one can see from the scope of this review, the pathogenesis of the vascular complications of diabetes is incredibly “complicated” as depicted by the number of pathways implicated. Therefore, it is not surprising that these disorders as a result of diabetes are named complications, given that the dictionary meaning of the word complicated is “something that is difficult to analyze or understand.”

Ghosh P., et al focused their attention to understand “The Role of Complement and Complement Regulatory Proteins in the Complications of Diabetes” and published the review in Endocr Rev. 2015 June; 36 (3): 272-288, the entire contents of which are incorporated herein by reference thereto. In the article, they summarized the body of evidence that supports a role for the complement system and complement regulatory proteins in the pathogenesis of diabetic vascular complications, with specific emphasis on the role of the membrane attack complex (MAC) and of CD59, an extracellular cell membrane-anchored Inhibitor of MAC formation that is inactivated by non enzymatic glycation. They discussed a pathogenic model of human diabetic complications in which a combination of CD59 inactivation by glycation and hyperglycemia-induced complement activation leading to increases Membrane Attack Complex (MAC) deposition that activates pathways of intracellular signaling, and induces the release of pro inflammatory, pro thrombotic cytokines and growth factors. Combined, complement-dependent and complement-independent mechanisms induced by high glucose promote inflammation, proliferation, and thrombosis as characteristically seen in the target organs of diabetes complications.

Tan S. M. Et al in “The Complement Pathway: New Insights into Immuno metabolic Signaling in Diabetic Kidney Disease” published in ANTIOXIDANTS & REDOX SIGNALING, Volume 37, Numbers 10-12, 2022, highlight the role of complement activation in causing Diabetic kidney disease, the entire contents of which are incorporated herein by reference thereto. The authors point out the critical issues that recent evidence indicate is that conventional reno protective agents used in DKD do not target the complement, leaving this web of inflammatory stimuli intact. They advise that the future studies should focus on the development of novel pharmacological agents that target the complement pathway to alleviate inflammation, oxidative stress, and kidney fibrosis, thereby reducing the burden of microvascular diseases in diabetes.

To understand the complex role of Complement System in Diabetes Complications—One should carefully study Complement system, as explained in this educational video link www.youtube.com/watch?v=BSypUV6QUNw. The video explains following key elements of complexity. Further information may be found in Rioklin D., et al, Complement component C3—The “Swiss Army Knife” of innate immunity and host defense, Immunol Rev. 2016 November; 274 (1): 33-58, the entire contents of which are incorporated herein by reference thereto.

The Complement system has evolved over 700 million years ago and is an army of over 30 different proteins, that work together in a complex and elegant dance to stop intruders and protect host.

All in all, about 15 quintillion complement proteins are saturating every fluid in our body. A healthy adult has about 5.3 micro molar of C3 in their blood. For this calculation, round it to 5. One micro molar equals 6.022×10\hat{ }17 molecules in a liter. An average adult contains roughly 5 liters of blood. So, 6×10\hat{ }17×5×5=1.5×10 \hat{ }19, or 15 quintillion.

One of the key players of our immune system is the complement system. An army of millions and trillions of tiny bombs, which work together in a complex and elegant dance to stop intruders in our body to protect host cells.

When the dysfunction of complement system occurs, what happens can be explained by the analogy “When one match stick is lit—the whole match box sets up the fire.” This leads to the explosion of millions and trillions of tiny bombs that are turned against our own host tissues damaging vascular systems in various organs such as brain, eyes, heart, kidney and peripheral blood vessels.

The Nobel prize winner Carolyn Bertozzi in 2022 identified normal host cells are highly glycosylated, see profiles.stanford.edu/carolyn-bertozzi.

Factor H is also highly glycosylated protein of 150 kD size that circulates in the body and bind to host cell glycosylated sites on one side. These cells are protected by Factor H from the host activated complement system. The details are found in the appendix under Factor H review.

In diabetes abnormal glycosylation pattern is normally seen in blood as high level of Hemoglobin Alc. But this type of abnormalities are also seen on host cell surface. This abnormal glycosylation pattern on cell surfaces contribute to factor H malfunction. This results in the dysfunction of complement system contributing to vascular complications of diabetes. An article describing this in greater detail is: Reily C. et al, Glycosylation in health and disease, Nature Reviews-Nephrology, Vol. 15, June 2019, 346-366, the entire contents of which are incorporated herein by reference thereto.

Our two decades long analysis of Factor H and how it relates to dysfunction of complement system is the subject of over 16550 scientific articles that are published in PubMed Central® (PMC). This is a free full-text archive of biomedical and life sciences journal literature at the U. S. National Institutes of Health's National Library of Medicine (NIH/NLM) and can be accessed easily by typing the word “Factor H” at PubMed central. www.ncbi.nlm.nih.gov/pmc/?term=Factor+H+. These articles review in depth to understand the role of Factor H in normal host and what abnormalities occur in the presence of various life threatening diseases.

Above contributes to our key understanding of genomic advances that Factor H is a dominant immune regulatory proteins of 150 kD that is located in chromosome 1 at Q32 position. This is a weakest link that is exploited by several microbes, viruses, cancer cells and age related diseases to contribute various major life threatening diseases.

Above genomic advance is also the key “Recent advance in “The Fundamentals of Immunology” as it is the key molecule that plays a dominant role in protecting host cell surface both in fluid and host cell surface.

We are advancing upon above “The Recent Advances in Genomics and Fundamentals of Immunology”. This has contributed to the understanding of how the C3 amplification loop results in dysfunction of the complement system contributing to over 80% inflammation and weaken immune system function.

Strategic exploitation of sulfonic polymers and its formulation strategies are outlined to provide immune prophylaxis and therapy for diabetes's complications with immune boosting.

Complement system is continuously active at a low level in the normal host, but damage to various organs is protected from complement activation due to the presence of complement regulators in blood and on host cell surface. This tightly regulate this spontaneous complement activation. Control of complement activation at the level of C3 convertase was sufficient to prevent complement-mediated inflammation in various organs.

This invention details the application of first principle to accelerate prophylaxis and therapy of diabetes complications with immune boosting.

A first principle is a basic assumption that cannot be deduced any further. Over two thousand years ago, Aristotle defined a first principle as “the first basis from which a thing is known.”

First principles thinking is a fancy way of saying “think like a scientist.” Scientists don't assume anything.

First principles thinking (also called reasoning from first principles) requires breaking down a problem into its fundamental building blocks, its essential elements, asking powerful questions, getting down to the basic truth, separating facts from assumptions and then constructing a view from the grounds up.

First principle or ‘knowledge-based’ models start from the basis of established science that are generally accepted and have been extensively verified.

This is applied to the study of complement system in the context of “Recent advances in genomics and fundamentals of immunology”. This has helped us to simplify the working of the system to key element that is Amplification loop of Complement system that control 80% of complement activation.

Identifying downstream effects of such complement activation that contribute to vascular complications of diabetes in various organs. This refers to C3a-C5a activations causing liberation of anaphylotoxins and its down steam interactions with C3aR and C5aR (R means Receptors) to stimulate inflammatory cytokines.

Developing the drug and formulation methods for blocking amplification ion loop of complement system.
Obtaining pre-clinical proof of concept data.
Developing human safety data.
Identifying most efficient regulatory pathway for quick clinical implementation of technology advances.

Above requires extensive literature review of “Recent Advances in the Fundamentals of Immunology and how it can be applied to prevent the vascular complications of diabetes.

Further, it also involved studying of the competitive molecular targeting of complement system as drug development process by biopharmaceutical industry and identifying key regulatory obstacles in drug development.

Immunity is a major barrier in the translational development of biopharmaceutical drugs. The human safety issue remains a problem that affects companies in both clinical trials and in post marketing phase. The drug industry's problems associated with meeting the regulatory safety and efficacy standards for new products are analyzed in an FDA report on innovation stagnation published in 2004 (FDA). The cost of successful product from bench to bed-side has soared to US $1.7 billion by 2002. The US Food and Drug Administration estimates a cost saving of US $100 Million if the human safety of new drugs could be predicted with 10% accuracy.

### SUMMARY OF THE INVENTION

It is the first object of the invention to identify molecular targets involved in “Complement activation Loop at C3 level”

It is second object of the invention to develop a drug and demonstrate proof of concept data by in-vitro studies in the laboratory setting. This is done by molecular targeting and kinetic study of such molecular targets of proteins of “C3 amplification ion loop”

It is the third object of the invention to develop a proof of concept data by in-vivo studies in animal models to show “C3 complement activation loop” can be inhibited and it will have therapeutic anti-inflammatory effects.

It is the fourth object of the invention to define the clinical safety of the drug at human level.

It is the fifth object of the invention to find the most efficient regulatory path “Right to Try” to meet the growing unmet medical needs for prophylaxis and therapy of vascular complications of diabetes.

These and other related objects are achieved according to a method for prophylaxis and therapy for vascular complications of diabetes along with immune boosting. In a first step, a pharmacological composition is formulated as a polystyrene sulfonate based pharmacological composition selected from the group consisting of sodium polystyrene sulfonate, calcium polystyrene sulfonate, formulation variations thereof, or combinations thereof. Next, a patient is selected that is immune compromised or at risk of or suffering vascular complications of diabetes. An effective amount of the pharmacological composition is administered to the patient. The pharmacological composition selectively targets key proteins involved in the C3 Amplification loop to provide prophylaxis, immune boosting and therapy for the vascular complications of diabetes.

The pharmacological composition selectively down regulates key proteins involved in the C3 amplification loop, wherein the key proteins include Factor B, Factor D and Factor H. The down regulation of key proteins involved in the C3 amplification loop contributes to cross talk inhibition of classical and lectin pathways. The cross talk inhibition limits C3a and C5a interactions with corresponding receptors of C3a and C5a. The cross talk inhibition triggers downstream inhibition of C5b-9 pathways and CD 59 pathways. The downstream inhibition further inhibits coagulation and thrombotic pathways that join at a C5 level.

The inhibition of both Factor H and Factor D enhances human safety by reducing adverse effects of inhibition of either Factor H or Factor D. The inhibition of both Factor H and Factor D enhances immunity of patients against microbes, viruses and cancer cells by targeting immune evasions and its adverse effects. The pharmacological composition further includes individually targeted complement inhibitors.

The formulating step further includes formulating a polystyrene sulfonate based pharmacological composition selected from the group consisting of fractional purified sodium polystyrene sulfonate, fractional purified calcium polystyrene sulfonate, or combinations thereof. The formulating step additionally includes formulating a polystyrene sulfonate based pharmacological composition selected from the group consisting of sodium polystyrene sulfonated nano polymer, calcium polystyrene sulfonated nano polymer, or combinations thereof.

In an alternate embodiment, the formulating step further includes formulating a polystyrene sulfonate based pharmacological composition selected from the group consisting of sodium polystyrene sulfonated nano polymer, calcium polystyrene sulfonated nano polymer, or combinations thereof. The formulating step additionally includes formulating a polystyrene sulfonate based pharmacological composition selected from the group consisting of fractional purified sodium polystyrene sulfonate, fractional purified calcium polystyrene sulfonate, or combinations thereof.

The administering step includes strategic administering to enhance the safety and efficacy to treat vascular complications of diabetes. The administering step further includes administering the pharmacological composition to the patient intravenously in a dose of 5-10 mg/kg body weight in 250 ml-500 ml 0.85% saline. The administering step includes administering the pharmacological composition to the patient orally in a dose of 5-10 mg/kg body weight with a pharmaceutically acceptable excipient. The administering step includes administering the pharmacological composition to the patient topically. The pharmacological composition is administered in combination with current and evolving diabetic drugs for treating vascular complications of diabetes.

### BRIEF DESCRIPTION OF THE DRAWINGS

These and other aspects, features and advantages of present invention will be described or become apparent from the following detailed description of the preferred embodiments, which is to be read in connections with the accompanying drawings.

FIG. 1 is a diagram showing the role of complement activation loop at C3 and how it contributes to vascular complications of diabetes.

FIG. 2 is a table showing Binding characteristics of complement proteins with polystyrene sulfonate

FIG. 3 shows In-vivo Mice efficacy data targeting the most inflammatory model of Type 1 Diabetes to cure diabetes in Non Obese Diabetes (NOD) mice.

### DETAILED DESCRIPTION OF THE PREFERRED EMBODIMENTS

These and other aspects, features and advantages of present invention will be described or become apparent from the following detailed description of the preferred embodiments, which is to be read in connections with the accompanying drawings.

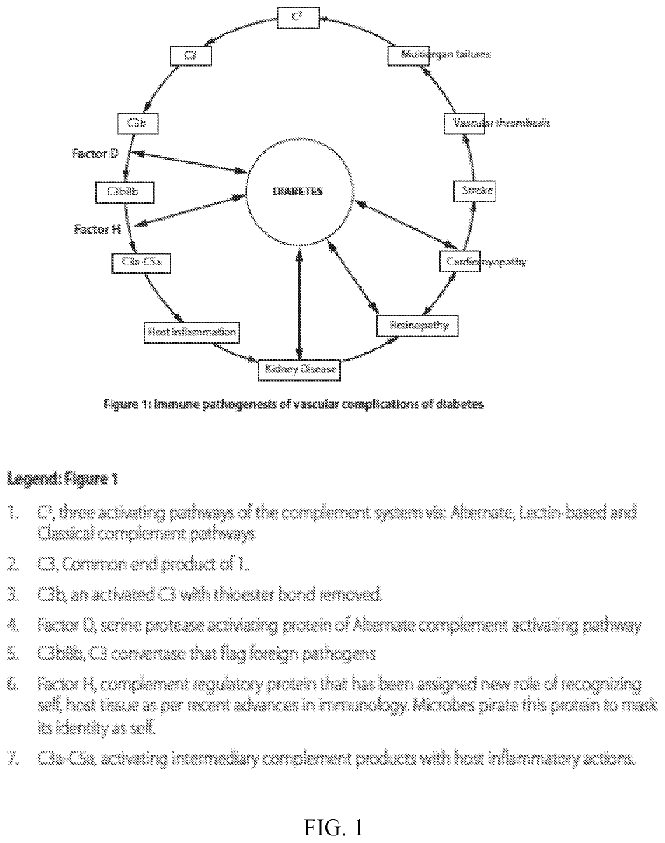

FIG. 1 explains the role of complement activation loop at C3 and how it contributes to vascular complications of diabetes.

C3 represents three activating pathways (e.g. Classical, Lectin and Alternate complement system).

C3 is the common end product of C 3 that is C3b

This is an activated C3 with thioester bond removed.

Factor D is a serine protease activating protein of alternate complement system, C3bBb is a C3 Convertase.

Factor H is a complement regulatory protein that has been assigned a new role of recognizing self, as per recent advances in genomics and fundamentals of immunology playing a dominant role over other immune regulatory molecules such as CD 59 present on host cell surface.

Factor H function is impaired due to abnormal glycosylation. This hyper activate “C3 Amplification loop” generating more and more C3bBb. C3a-C5a is anaphylotoxin and breakdown product of C3bBb that reacts with receptors of C3aR and C5aR (Not shown) to stimulate production of inflammatory cytokines. They contribute to vascular complications of diabetes.

FIG. 1—Immune Pathogenesis of Vascular complications of diabetes

### Legend: FIG. 1

1. C3, three activating pathways of the complement system vis: Alternate, Lectin-based and Classical complement pathways.
2. C3, Common end product of 1.
3. C3b, an activated C3 with thioester bond removed.
4. Factor D, serine protease activating protein of Alternate complement activating pathway.
5. C3bBb, C3 convertase that flags foreign pathogens.
6. Factor H, complement regulatory protein that has been assigned new role of recognizing self, host issue as per recent advances in immunology. Microbes pirate this protein to mask its identity as self.
7. C3a-C5a, activating intermediary complement products with host inflammatory actions.

Above contributing to Vascular complications of Diabetes affecting multiple organs Kidney/Eyes/Cardiac/Brain/Peripheral Blood vessels/Multiple organ failures

Notes: Not shown above are:

a. Host Inflammation: This is due to C3a-C5a interactions
b. Fatty Acids: Immune metabolic signals are generated by C5a interacting with C5aR receptors
c. Fibrotic responses: This is generated due to C3a-C5a interacting with host cell immune receptors to release TGF signals
d. Coagulation pathway meets complement cycle after C5a formation to cause thrombotic complications
e. Cytotoxic immune responses are due to C5b-C9 activation on host cell surfaces to form membrane attack complex
f. Insulin resistance is due to over activation of both Factor H and Factor D reducing Beta cell function.

As shown in FIG. 1 above, the immune pathogensis of vascular diabetes complications are due to abnormal glycation in the blood and host cells. Their interactions with C3 activation loop contribute to more than 80% of complement activation. This depiction of vascular complications of diabetes is in keeping with Recent Advances in Genomics and Immunology.

The above objectives are met as follows:

Endocrine Technology, LLC is NY based Biotechnology Company established on Feb. 12, 2002. As a physician based biotechnology company-we actively treat large number of patients with diabetes and its complications.

Our initial efforts were directed for the cure of Type 1 Diabetes in early 1990s. While working at a largest transplant center in Asia for “Kidney diseases” in Ahmedabad, G. S., India, we have noticed that the success of hemodialysis in the treatment of “chronic Renal Failure” was due to major improvements in “hollow fibers used for Hemodialysis”. This prevented the occurrence of life threatening “acute respiratory failure” which was common place earlier.

We capitalized on these advances to develop technology to target “Amplification of complement loop at C3 level”

We present preclinical proof of concept data to modulate “Amplification of complement loop at C3 level” by molecular targeting of Factor D, Factor H and Factor B. These are the key components involved in C3 mediated amplification loop of complement system. Classical and left mediated activation is also amplified by C3 amplification loop. They are inhibited secondarily. Tertiary inhibition of thrombin mediated path occur as it joins the distal part of complement activation axis.

Identification of Key Molecular Targets: A careful perusal of Recent Advances in Complement System suggest that Alternate Complement System is the key driver of Immune pathogenesis of vascular diseases in diabetes where there is proximal complement system activation. A further paper describing the interaction between the complement system and vascular disease is: de Boer et al, Therapeutic Lessons to be Learned From the Role of Complement Regulators as Double-Edged Sword in Health and Disease, Frontiers in Immunology, December 2020, Vol. 11, Article 578069, the entire contents of which are incorporated herein by reference thereto.

Factor D is a 25 kD fluid protein of Alternate Complement System. It is a key driver of proximal complement activation. The relentlessly activated proximal complement system cause down stream activation of inflammatory mediators that is key driver of vascular inflammation and its complications.

Factor H is a complement regulatory protein that has been assigned a new role of recognizing self, as per recent advances in genomics and fundamentals of immunology playing a dominant role over other immune regulatory molecules present on host cell surface.

Factor H is mutated in diabetic retinopathy, the cell surface glycosylation is abnormal and in corneal injuries there is damage to immune regulatory molecules including Factor H. These factors hyper activate “C3 Amplification loop” generating more and more C3bBb. C3a-C5a is anaphylotoxin and its breakdown product of C3bBb that reacts with receptors of C3a and C5a to stimulate production of inflammatory cytokines. They contribute to vascular inflammation and eventually leads to complications.

The binding of Complement amplification loop proteins at C3 level was studied for Factor B, Factor D, Factor H as well as for Factor I proteins. The studies involved binding kinetics of sulfonic nano polymer and other polymers. NHS with buffer was used as normal control. The dose of polymers used was standardized.

The NHS in (1:4) dilution with each of above polymers was incubated at 37° C. in the water bath with gentle agitation for half hour to 1 hour durations in a series of experiments. The binding activity of complement proteins in the serum was analyzed in duplicate and mean result is depicted. Below are the key results.

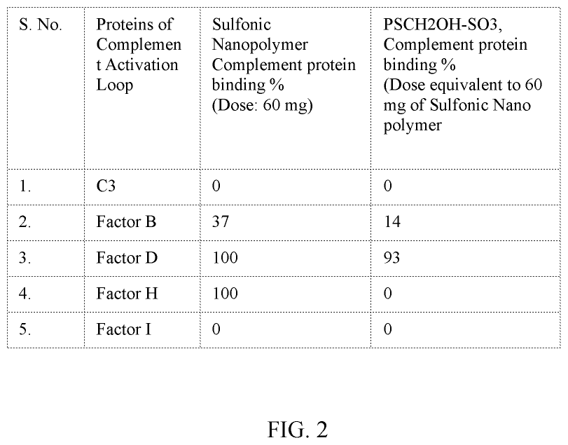

FIG. 2 is a table showing Binding characteristics of complement proteins with polystyrene sulfonate.

In FIG. 2, Assay incubation time was 60 min at 37° C., sulfonic nano polymer used was (60 mg=1056 cm²) and PSCH2OH—SO3 130 mg=1092 cm². The complement protein concentrations were measured in the supernatant and the specific adsorption was determined from the loss of protein observed in the presence of polymers as compared to control incubation.

In-Vitro Data: A. The drug application is based on the fact that as shown above it is a complement inhibitor of Factor D that will inhibit
a. down stream interactions with C3a-5a that contribute to inflammatory interactions
b. C3a-C5a interacts with C3aR and C5aR receptors to stimulate immuno metabolic or fatty acid synthesis signals
c. C3a-C5a also interacts with abnormal glycosylation on host cells to stimulate TGF signals causing fibrotic interactions
d. Formation of C5b-C9 signals on host cells form membrane attack complex (MAC) and interactions with CD 59 receptors for inflammatory and cytotoxic signals.

In-vitro Data: B. The drug also inhibits Factor H. As shown above, Factor H is a dominant immune regulatory molecule of Alternate complement system that protect host in both at fluid and over cell membrane. This protein is mutated in diabetes causing complement activation and amplifying inflammatory cascades.

In-vivo mice data: It inhibit Complement system when the drug is given intravenously at 0.5 mg/kg body weight leading to inhibition of CH50 activity.

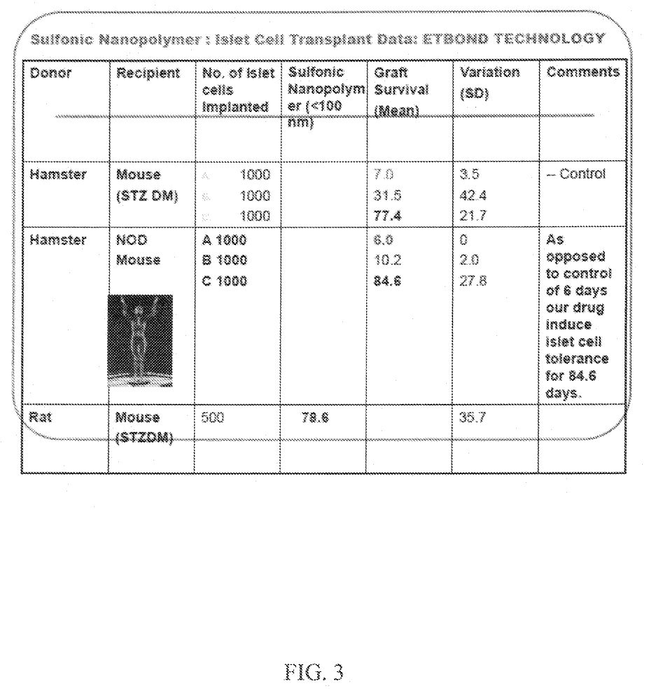

FIG. 3 shows In-vivo Mice efficacy data in the most inflammatory model of Type 1 Diabetes. It has been used successfully to inhibit inflammation in small animal models including Non Obese Diabetes (N.O.D.) Mice leading to the cure of diabetes.

### Details of Polystyrene Sulfonate

HISTORY: PSS is an endocrine drug originally introduced to medicine in 1935 and was formally approved in the USA in 1975. It has been used since 1975 for the therapy of high potassium. (Gerstman B. B. and Platt R. Uses of sodium polystyrene sulfonate in sorbitol in the United States, 1985-89. A J. of Kidney Dis., 1991:15, No. 5, 619-620, the entire contents of which are incorporated herein by reference thereto; Sodium polystyrene sulfonate, Martindale: The complete Drug Reference, 32nd edition, edited by Parfitt K., Pharmaceutical Press, 1999:995-996), the entire contents of which are incorporated herein by reference thereto. Its ability to manipulate immune system, provide antibacterial and antitoxin effects have overlapping basic principles defined in endocrinology and immunology. Ideas and observations from clinics can be brought to the laboratory or to a more basic level of translational research hierarchy for further investigation as shown by the new applications of an established drug.

PSS is a globally available drug at cost effective prices. (Sodium polystyrene sulfonate, Martindale: The complete Drug Reference, 32nd edition, edited by Parfitt K., Pharmaceutical Press, 1999:995-996). The mean approved dosage for the therapy of established indications, such as the therapy of high potassium, is 90 grams per day.

Based on the immune modulation data reported in-vitro and in-vivo, the dose required appears to be less than a gram per day. The drug is preferably given in the dose of 5-10 mg/kg body weight. It is given as intravenous infusion in 250 ml-500 ml 0.85% saline over 2-3 hours to permit action of this drug at tissue or blood level where it is needed most.

The frequency of doses could be once or twice a day. The mucosal safety data and blood contact safety data is readily available at this dosage. (Setoyama H., Inoue K., Iwata H. et al. The Potential of Anti-Complement Synthetic Sulfonic Polymers for Xenotransplantation. Transplantation Proceedings, 1998, 30:67-70, the entire contents of which are incorporated herein by reference thereto; Gerstman B. B. and Platt R. Uses of sodium polystyrene sulfonate in sorbitol in the United States, 1985-89. A J. of Kidney Dis., 1991:15, No. 5, 619-620, the entire contents of which are incorporated herein by reference thereto; Sodium polystyrene sulfonate, Martindale: The complete Drug Reference, 32nd edition, edited by Parfitt K., Pharmaceutical Press, 1999:995-996; Inaba S., Nibu K., Takano H. et al. Potassium adsorption filter for REC transfusion: A phase iii clinical trial. Transfusion, 2000; 40:1469-1474), the entire contents of which are incorporated herein by reference thereto.

According to the FDA, a new formulation represents the unlabeled use of an established drug. FDA regulation permits the use of such drug by a licensed physician in life threatening infections. According to FDA commissioner Charles C. Edwards, “Once the new drug is in pharmacy, the physician may, as part of the practice of medicine vary the condition of use from those approved in the package insert, without obtaining approval of the FDA” as per The Federal Registrar, Vol. 37, No. 158, Aug. 15, 1972. This is clearly restated in the April 1982 FDA Drug Bulletin. Careful perusal of the current IND document also makes this clear. (Food and Drug Administration, HHS, Part 312-Investigational New Drug Application). As a prophylaxis and therapy of vascular complications, the present invention advantageously comprises a new application of an established drug that can be immediately used with zero tolerance to time. This will be life saving, prevent malpractice risk and impact favorably in reducing the economic costs by reducing vascular complications of diabetes. The risk management involves carefully monitoring adverse effects in the patient and discontinuing the drug if adverse effects are observed.

Although illustrative embodiments of the present invention have been described herein with reference to the accompanying drawings, it is to be understood that the present invention is not limited to those precise embodiments, and that various other changes and modifications may be affected therein by one skilled in the art without departing from the scope or spirit of the invention. All such changes and modifications are intended to be included within the scope of the invention as defined by the appended claims.

What is claimed is:

1. A method for prophylaxis and therapy for vascular complications of diabetes with immune boosting comprising the steps of:
formulating a polystyrene sulfonate based pharmacological composition selected from the group consisting of sodium polystyrene sulfonate, calcium polystyrene sulfonate, formulation variations thereof, or combinations thereof;
selecting a patient that is immune compromised or at risk of or suffering vascular complications of diabetes;
administering of an effective amount of the pharmacological composition to the patient; and
wherein the pharmacological composition selectively targets key proteins involved in the C3 Amplification loop to provide prophylaxis, immune boosting and therapy for the vascular complications of diabetes.

2. The method of claim 1, wherein the pharmacological composition selectively down regulates key proteins involved in the C3 amplification loop, wherein the key proteins include Factor B, Factor D and Factor H.

3. The method of claim 2, wherein the down regulation of key proteins involved in the C3 amplification loop contributes to cross talk inhibition of classical and lectin pathways.

4. The method of claim 3, where cross talk inhibition limits C3a and C5a interactions with corresponding receptors of C3a and C5a.

5. The method of claim 4, wherein the cross talk inhibition triggers downstream inhibition of C5b-9 pathways and CD 59 pathways.

6. The method of claim 5, where the downstream inhibition further inhibits coagulation and thrombotic pathways that join at a C5 level.

7. The method of claim 1, where inhibition of both Factor H and Factor D enhances human safety by reducing adverse effects of inhibition of either Factor H or Factor D.

8. The method of claim 7, where inhibition of both Factor H and Factor D enhances immunity of patients against microbes, viruses and cancer cells by targeting immune evasions and its adverse effects.

9. The method of claim 1, wherein the pharmacological composition further includes individually targeted complement inhibitors.

10. The method of claim 1, wherein the formulating step includes:
formulating a polystyrene sulfonate based pharmacological composition selected from the group consisting of fractional purified sodium polystyrene sulfonate, fractional purified calcium polystyrene sulfonate, or combinations thereof.

11. The method of claim 10, wherein the formulating step includes:
formulating a polystyrene sulfonate based pharmacological composition selected from the group consisting of sodium polystyrene sulfonated nano polymer, calcium polystyrene sulfonated nano polymer, or combinations thereof.

12. The method of claim 1, wherein the formulating step includes:
formulating a polystyrene sulfonate based pharmacological composition selected from the group consisting of sodium polystyrene sulfonated nano polymer, calcium polystyrene sulfonated nano polymer, or combinations thereof.

13. The method of claim 12, wherein the formulating step includes:
formulating a polystyrene sulfonate based pharmacological composition selected from the group consisting of fractional purified sodium polystyrene sulfonate, fractional purified calcium polystyrene sulfonate, or combinations thereof.

14. The method of claim 1, wherein the administering step includes strategic administering to enhance the safety and efficacy to treat vascular complications of diabetes.

15. The method of claim 1, wherein the administering step includes:
administering the pharmacological composition to the patient intravenously in a dose of 5-10 mg/kg body weight in 250 ml-500 ml 0.85% saline.

16. The method of claim 1, wherein the administering step includes:
administering the pharmacological composition to the patient orally in a dose of 5-10 mg/kg body weight with a pharmaceutically acceptable excipient.

17. The method of claim 1, wherein the administering step includes:
administering the pharmacological composition to the patient topically.

18. The method of claim 1, wherein the pharmacological composition is administered in combination with current and evolving diabetic drugs for treating vascular complications of diabetes.
